(19) United States
(12) Patent Application Publication
(10) Pub. No.: US 2025/0320309 A1
(43) Pub. Date: Oct. 16, 2025

(54) BISPECIFIC ANTIGEN BINDING PROTEIN

(71) Applicant: SHENGHE (CHINA) BIOPHARMACEUTICAL CO., LTD., Nanjing, Jiangsu (CN)

(72) Inventors: Xiaoling JIANG, Nanjing (CN); Chunling QIN, Nanjing (CN); Chong ZHOU, Nanjing (CN); Chongbing WU, Nanjing (CN); Liusong YIN, Nanjing (CN)

(73) Assignee: SHENGHE (CHINA) BIOPHARMACEUTICAL CO., LTD., Nanjing, Jiangsu (CN)

(21) Appl. No.: 18/713,398

(22) PCT Filed: Nov. 23, 2022
(86) PCT No.: PCT/CN2022/133618

§ 371 Date: May 24, 2024

(30) Foreign Application Priority Data
Nov. 25, 2021 (WO) PCT/CN2021/132937

(51) Int. Cl. C07K 16/28; A61K 39/00; A61P 35/00

(52) U.S. Cl. CPC C07K 16/2896; A61P 35/00; C07K 16/2878; A61K 2039/505; C07K 2317/31; C07K 2317/732; C07K 2317/92

(57) ABSTRACT

A bispecific antigen binding protein, including (a) a first antibody or antigen binding fragment thereof that specifically binds to a first antigen; and (b) a second antibody or antigen binding fragment thereof that specifically binds to a second antigen. In the embodiments, the first antigen is CD24, and the second antigen is 4-1BB; or, the first antigen is 4-1BB, and the second antigen is CD24.

### REFERENCE TO A SEQUENCE LISTING

In accordance with 37 CFR § 1.831, the present specification makes reference to a Sequence Listing submitted electronically as a .xml file named “SH20240508.xml”. The .xml file was generated on Oct. 16, 2024, and is 61,184 bytes in size. The entire contents of the Sequence Listing are hereby incorporated by reference.

### TECHNICAL FIELD

The present invention relates to the field of tumor immunotherapy and molecular immunology, and specifically to a bispecific antigen binding protein that specifically binds to CD24 and 4-1BB.

### BACKGROUND

Tumor-targeted monoclonal antibody is one of the important methods in the field of tumor immunotherapy. Macrophages need two signals to act simultaneously to exert phagocytosis: One is the activation of the “eat me” signal on the surface of the target cell, and the other is the inactivation of the “don't eat me” signal on the surface of the same cell. The absence of any one of these signals is not sufficient to trigger phagocytosis. CD24 is a class of “don't-eat-me” signals, which are highly expressed by tumor cells and released by binding to Siglec-10 on the surface of macrophages, thus preventing tumor cells from being phagocytosed by macrophages.

4-1BB (CD137, TNFRSF9) is a transmembrane protein of the tumor necrosis factor receptor superfamily (TNFRS). 4-1BB is expressed as a monomer or dimer on the cell surface and binds to its ligand (4-1BBL) through trimerization for signaling, and is a co-stimulatory molecule for CD8+ and CD4+ T cells, regulatory T cells (Tregs), NK cells and NKT cells, B cells, and neutrophils, among others. On T cells, 4-1BB is not constitutively expressed, but is induced upon T cell receptor (TCR) activation and signaling via TNFR-associated factor (TRAF)-2 and TRAF-1 via stimulation by its natural ligand 4-1BBL or antibody agonists. Early signaling by 4-1BB involves polyubiquitination of K-63, activation of the nuclear factor (NF)-κB and mitogen-activated protein kinase (MAPK) pathways, and signaling leading to co-stimulation of T-cells, cell proliferation, cytokine production, and prolonged survival of mature and CD8+ T-cells.

There are no reports in the prior art of bispecific antigen binding proteins that can specifically bind both 4-1BB and CD24, and the present invention is the first to develop a bispecific antigen binding protein that binds to 4-1BB to CD24. This bispecific antigen binding protein has the following advantages at the same time: it can specifically recognize CD24 and 4-1BB; it can effectively exert ADCC effects and ADCP effects on CD24-expressing tumor cells through NK cells and macrophages; It can depend on the cross-linking effect of anti-CD24 end, specifically activate the signaling pathway of 4-1BB, activate CD8+ T cells, which can stimulate the proliferation of T cells and secretion of cytokines, specifically kill CD24-expressing tumor cells, and improve the anti-tumor effect of the body; It can also kill regulatory T cells (Tregs) with high expression of 4-1BB through ADCC activity, lift the immunosuppression of the tumor microenvironment, and promote the infiltration of CD8+ T cells to achieve anti-tumor effects.

The bispecific antigen binding protein specifically recognizes CD24 and 4-1BB; it can effectively exert ADCC effects and ADCP effects on CD24-expressing tumor cells via NK cells and macrophages; It can depend on the cross-linking effect of anti-CD24 end, specifically activate the signaling pathway of 4-1BB, activate CD8+ T cells, which can stimulate the proliferation of T cells and secretion of cytokines, specifically kill CD24-expressing tumor cells, and improve the anti-tumor effect of the body; It can also kill regulatory T cells (Tregs) with high expression of 4-1BB through ADCC activity, release the immunosuppression of the tumor microenvironment, and promote the infiltration of CD8+ T cells to achieve anti-tumor effects.

### SUMMARY

The present invention provides a bispecific antigen binding protein, comprising:
(a) a first antibody or antigen binding fragment thereof that specifically binds to a first antigen; and
(b) a second antibody or antigen binding fragment thereof that specifically binds to a second antigen;
wherein:
the first antigen is CD24, and the second antigen is 4-1BB; or, the first antigen is 4-1BB, and the second antigen is CD24.

In alternative embodiments, the first antibody or antigen binding fragment thereof comprises a heavy chain and a light chain; and
the second antibody or antigen binding fragment thereof comprises a scFv;
wherein, the scFv is linked to the N-terminal or C-terminal end of the heavy or light chain of the first antibody or antigen binding fragment thereof.

In alternative embodiments, the heavy chain variable region of one heavy chain of the first antibody or antigen binding fragment thereof forms an antigen binding site with the light chain variable region of one light chain, and the heavy chain variable region of another heavy chain forms an antigen binding site with the light chain variable region of another light chain.

In alternative embodiments, the bispecific antigen binding protein comprises one first antibody or antigen binding fragment thereof and one or more scFv(s).

In alternative embodiments, the bispecific antigen binding protein comprises one first antibody or antigen binding fragment thereof and two scFvs.

In alternative embodiments, one scFv is linked to the N-terminal end of the heavy or light chain of the first antibody or antigen binding fragment thereof, and the other scFv is linked to the C-terminal end of the heavy or light chain of the first antibody or antigen binding fragment thereof.

In alternative embodiments, two scFvs are linked to the N-terminal ends of two heavy chains or two light chains of the first antibody or the antigen binding fragment thereof, respectively; or, two scFvs are linked to the C-terminal ends of two heavy chains or two light chains of the first antibody or the antigen binding fragment thereof, respectively.

In alternative embodiments, the bispecific antigen binding protein comprises two first polypeptide chains and two second polypeptide chains, for each of the polypeptide chains:
(a) the first polypeptide chain each independently comprises the heavy chain of the first antibody or antigen binding fragment thereof and the scFv; and
(b) the second polypeptide chain each independently comprises the light chain of the first antibody or antigen binding fragment thereof.

In alternative embodiments, the bispecific antigen binding protein comprises two first polypeptide chains and two second polypeptide chains, for each of the polypeptide chains:
(a) the first polypeptide chain each independently comprises the heavy chain of the first antibody or antigen binding fragment thereof; and
(b) the second polypeptide chain each independently comprises the light chain of the first antibody or antigen binding fragment thereof and the scFv.

In alternative embodiments, two first polypeptide chains are the same or different, and/or two second polypeptide chains are the same or different.

In alternative embodiments, the heavy chain variable region of the scFv is linked to the light chain variable region by a linker L1, and/or two scFvs are linked to the N-terminal or C-terminal ends of two heavy chains or two light chains of the first antibody or antigen binding fragment thereof, respectively, by a linker L2.

In alternative embodiments, the linker L1 and the linker L2 are the same or different.

In alternative embodiments, the linker L1 and/or the linker L2 have an amino acid sequence as shown in (G₄S)ₓ, x is an integer selected from 1-6; preferably, the linker L1 and/or L2 is (G₄S)₃ or (G₄S)₄.

In alternative embodiments, a disulfide bond exists between the heavy chain variable region and the light chain variable region of the scFv.

In alternative embodiments, the heavy chain of the first antibody or antigen binding fragment thereof comprises a heavy chain variable region and a heavy chain constant region, and the light chain comprises a light chain variable region and a light chain constant region; preferably, the first antibody or antigen binding fragment thereof is a full-length antibody.

In alternative embodiments, the heavy chain of the first antibody or antigen binding fragment thereof comprises a first Fc region and a second Fc region.

In alternative embodiments, the first Fc region and the second Fc region are the same Fc or different Fc.

In alternative embodiments, the Fc region is selected from the group consisting of: IgG, IgA, IgD, IgE and/or IgM.

In alternative embodiments, the Fc region is selected from the group consisting of: IgG1, IgG2, IgG3 and/or IgG4.

In alternative embodiments, the first antibody or antigen binding fragment thereof specifically binds to CD24, and the scFv specifically binds to 4-1BB, wherein,
the first antibody or antigen binding fragment thereof comprises:
(a) HCDR1 as shown in SEQ ID NO: 2, HCDR2 as shown in SEQ ID NO: 3, HCDR3 as shown in SEQ ID NO: 4; and, LCDR1 as shown in SEQ ID NO: 6, LCDR2 as shown in SEQ ID NO: 7, and LCDR3 as shown in SEQ ID NO: 8; or
(b) HCDR1 as shown in SEQ ID NO: 10, HCDR2 as shown in SEQ ID NO: 11, and HCDR3 as shown in SEQ ID NO: 12; and, LCDR1 as shown in SEQ ID NO: 14, LCDR2 as shown in SEQ ID NO: 15, and LCDR3 as shown in SEQ ID NO: 16;
and, the scFv comprises:
HCDR1 as shown in SEQ ID NO: 18, HCDR2 as shown in SEQ ID NO: 19, and HCDR3 as shown in SEQ ID NO: 20; and, LCDR1 as shown in SEQ ID NO: 22, LCDR2 as shown in SEQ ID NO: 23, and LCDR3 as shown in SEQ ID NO: 24.

In alternative embodiments, the first antibody or antigen binding fragment thereof comprises:
(a) a heavy chain variable region VH as shown in SEQ ID NO: 1, and a light chain variable region VL as shown in SEQ ID NO: 5; or
(b) a heavy chain variable region VH as shown in SEQ ID NO: 9, and a light chain variable region VL as shown in SEQ ID NO: 13;
and, the scFv comprises:
(a) a heavy chain variable region VH as shown in SEQ ID NO: 17, and a light chain variable region VL as shown in SEQ ID NO: 21.

In alternative embodiments, the first antibody or antigen binding fragment thereof specifically binds to 4-1BB, and the scFv specifically binds to CD24, wherein,
the first antibody or antigen binding fragment thereof comprises:
HCDR1 as shown in SEQ ID NO: 18, HCDR2 as shown in SEQ ID NO: 19, HCDR3 as shown in SEQ ID NO: 20; and, LCDR1 as shown in SEQ ID NO: 22, LCDR2 as shown in SEQ ID NO: 23, and LCDR3 as shown in SEQ ID NO: 24;
and, the scFv comprises:
(a) HCDR1 as shown in SEQ ID NO: 2, HCDR2 as shown in SEQ ID NO: 3, HCDR3 as shown in SEQ ID NO: 4; and, LCDR1 as shown in SEQ ID NO: 6, LCDR2 as shown in SEQ ID NO: 7, and LCDR3 as shown in SEQ ID NO: 8; or
(b) HCDR1 as shown in SEQ ID NO: 10, HCDR2 as shown in SEQ ID NO: 11, HCDR3 as shown in SEQ ID NO: 12; and, LCDR1 as shown in SEQ ID NO: 14, LCDR2 as shown in SEQ ID NO: 15, and LCDR3 as shown in SEQ ID NO: 16.

In alternative embodiments, the first antibody or antigen binding fragment thereof comprises:
a heavy chain variable region VH as shown in SEQ ID NO: 17, and a light chain variable region VL as shown in SEQ ID NO: 21;
and, the scFv comprises:
(a) a heavy chain variable region VH as shown in SEQ ID NO: 1, and a light chain variable region VL as shown in SEQ ID NO: 5; or
(b) a heavy chain variable region VH as shown in SEQ ID NO: 9, and a light chain variable region VL as shown in SEQ ID NO: 13.

In alternative embodiments, the bispecific antigen binding protein comprises:
(1) a first polypeptide chain as shown in SEQ ID NO: 25, and a second polypeptide chain as shown in SEQ ID NO: 35;
(2) a first polypeptide chain as shown in SEQ ID NO: 25, and a second polypeptide chain as shown in SEQ ID NO: 36;
(3) a first polypeptide chain as shown in SEQ ID NO: 27, and a second polypeptide chain as shown in SEQ ID NO: 33;
(4) a first polypeptide chain as shown in SEQ ID NO: 28, and a second polypeptide chain as shown in SEQ ID NO: 33;
(5) a first polypeptide chain as shown in SEQ ID NO: 29, and a second polypeptide chain as shown in SEQ ID NO: 34;
(6) a first polypeptide chain as shown in SEQ ID NO: 30, and a second polypeptide chain as shown in SEQ ID NO: 34;
(7) a first polypeptide chain as shown in SEQ ID NO: 31, and a second polypeptide chain as shown in SEQ ID NO: 34;
(8) a first polypeptide chain as shown in SEQ ID NO: 32, and a second polypeptide chain as shown in SEQ ID NO: 34;
(9) a first polypeptide chain as shown in SEQ ID NO: 26, and a second polypeptide chain as shown in SEQ ID NO: 37;
(10) a first polypeptide chain as shown in SEQ ID NO: 26, and a second polypeptide chain as shown in SEQ ID NO: 38;
(11) a first polypeptide chain as shown in SEQ ID NO: 39, and a second polypeptide chain as shown in SEQ ID NO: 45;
(12) a first polypeptide chain as shown in SEQ ID NO: 40, and a second polypeptide chain as shown in SEQ ID NO: 45;
(13) a first polypeptide chain as shown in SEQ ID NO: 41, and a second polypeptide chain as shown in SEQ ID NO: 46;
(14) a first polypeptide chain as shown in SEQ ID NO: 42, and a second polypeptide chain as shown in SEQ ID NO: 46;
(15) a first polypeptide chain as shown in SEQ ID NO: 43, and a second polypeptide chain as shown in SEQ ID NO: 47; or
(16) a first polypeptide chain as shown in SEQ ID NO: 44, and a second polypeptide chain as shown in SEQ ID NO: 47.

The present invention also provides an isolated nucleic acid molecule comprising a nucleotide sequence encoding a bispecific antigen binding protein as described in any of the above;

Preferably, the isolated nucleic acid molecule comprises a nucleotide sequence encoding a first polypeptide chain of a bispecific antigen binding protein as described in any of the above;

Preferably, the isolated nucleic acid molecule comprises a nucleotide sequence encoding a second polypeptide chain of a bispecific antigen binding protein as described in any of the above.

The present invention also provides a recombinant vector comprising the isolated nucleic acid molecule as described above.

The present invention also provides a recombinant cell comprising the isolated nucleic acid molecule as described above and/or the recombinant vector as described above.

The present invention also provides the use of the bispecific antigen binding protein, the nucleic acid molecule, the recombinant vector and/or the recombinant cell in the preparation of a drug for the treatment and/or prevention and/or diagnosis of a disease.

The present invention also provides the use of the bispecific antigen binding protein, the nucleic acid molecule, the recombinant vector, and/or the recombinant cell in the preparation of a drug for the treatment of cancer.

### Terms

The term “bispecific antigen binding protein” refers to a protein molecule that specifically binds to two target antigens or target antigen epitopes. In the present invention, the term “bispecific antigen binding protein” comprising an antibody or antigen binding fragment (e.g, Fab, scFv, etc.) is used interchangeably with the terms “bispecific antibody” and “bis-antibody”

The term “antibody” usually refers to an immunoglobulin molecule consisting of two pairs of polypeptide chains (each pair having a light chain and a heavy chain). Heavy chains can be classified as μ, δ, γ, α, or ε, and antibody isotypes are defined as IgM, IgD, IgG, IgA, and IgE, respectively. Antibody light chains can be classified as κ and κ light chains. Each heavy chain consists of a heavy chain variable region (VH) and a heavy chain constant region (CH), and the heavy chain constant region consists of three structural domains (CH1, CH2 and CH3). Each light chain consists of a light chain variable region (VL) and a light chain constant region (CL), and the light chain constant region consists of one structural domain, CL. The constant structural domains are not directly involved in antibody-antigen binding but exhibit a variety of effector functions. The VH and VL regions can also be subdivided into regions with high variability (called complementary determining regions (CDRs)), with more conserved regions known as framing regions (FRs) scattered in between. Each VH and VL consists of three CDRs and four FRs arranged from amino-terminal to carboxy-terminal in the following order: FR1, CDR1, FR2, CDR2, FR3, CDR3, FR4. The variable regions of each heavy chain/light chain pair (VH and VL) form the antigen binding site, respectively.

The term “complementarity determining region” or “CDR” refers to one of six highly variable regions within the variable structural domain of an antibody that primarily contribute to antigen binding. One of the most commonly used definitions of the six CDRs described is provided by Kabat E. A. et al, ((1991) Sequences of proteins of immunological interest. NIH Publication 91-3242). As used in some embodiments herein, CDRs may be defined in terms of Kabat rules for CDR1, CDR2, and CDR3 (LCDR1, LCDR2, LCDR3) for light chain variable structure domains, and CDR1, CDR2, and CDR3 (HCDR1, HCDR2, HCDR3) for heavy chain variable structure domains; as used in some embodiments herein, CDRs may also be defined in terms of IMGT for CDR1, CDR2, and CDR3 of light chain variable structure domains (LCDR1, LCDR2, LCDR3), and for CDR1, CDR2, and CDR3 of heavy chain variable structure domains (HCDR1, HCDR2, HCDR3).

The term “antibody” includes, but is not limited to, monoclonal antibody, mouse antibody, camel antibody, chimeric antibody, humanized antibody, fully human antibody, bispecific or multispecific antibody. These antibodies may belong to any isotype/type (e.g., IgG, IgE, IgM, IgD, IgA, and IgY) or subclass (e.g., IgG1, IgG2, IgG3, IgG4, IgA1, and IgA2).

The term “antigen binding fragment” or “antigen binding portion” refers to one or more portions of an antibody that retain the ability to bind the antigen bound by the antibody. In some embodiments, an “antigen binding fragment” of an antibody comprises (1) a Fab fragment, a monovalent fragment comprising the structural domains of VL, VH, CL, and CH1; (2) F(ab′)₂ fragment, a bivalent fragment containing two Fab fragments linked by a disulfide bridge in the hinge region; (3) scFv fragment, consisting of the VL and VH structural domains of the antibody or of the VH and VL structural domains of the antibody; and (4) CDR, after isolation of the complementation determining region.

The term “polypeptide” refers to a chain of amino acids of any length, irrespective of modification (e.g. phosphorylation or glycosylation). The term polypeptide includes proteins and fragments thereof. Polypeptides can be “exogenous”, meaning that they are “heterologous”, i.e., foreign to the host cell being utilized, e.g., human polypeptides produced by bacterial cells. Polypeptides are disclosed herein as sequences of amino acid residues. Those sequences are written from left to right in the direction from the amino terminus to the carboxy terminus. According to standard nomenclature, amino acid residue sequences are named in three-letter or single-letter codes as follows: alanine (Ala, A), Arginine (Arg, R), Asparagine (Asn, N), Aspartic acid (Asp, D), Cysteine (Cys, C), Glutamine (Gln, Q), Glutamic acid (Glu, E), Glycine (Gly, G), Histidine (His, H), Isoleucine (Ile, I), Leucine (Leu, L), Lysine (Lys, K), Methionine (Met, M), phenylalanine (Phe, F), proline (Pro, P), serine (Ser, S), threonine (Thr, T), tryptophan (Trp, W), tyrosine (Tyr, Y) and valine (Val, V). Numbering of amino acid positions SEQ ID NO: 2-4 (e.g., amino acid residues in the Fc region) and target regions (e.g., CDR) as described herein was performed using the IMGT system, and numbering of amino acid positions SEQ ID NO: 1 and SEQ ID NO: 5-53 (e.g., amino acid residues in the Fc region) and target regions (e.g., CDR) was performed using the Kabat system.

The term “scFv” refers to a molecule comprising an antibody heavy chain variable structural domain (VH) and an antibody light chain variable structural domain (VL) linked by a linker. Such scFv molecules may have the general structure: NH2-VL-linker-VH-COOH or NH2-VH-linker-VL-COOH. suitable prior art linkers comprise repeated GGGGS amino acid sequences or variants thereof, e.g., by using 1-6 repeated GGGGS amino acid sequences or variants thereof.

The term “host cell” refers to a cell that has been or is capable of being transformed with a nucleic acid sequence and thus expressing the selected target gene. The term includes the progeny of a parental cell, whether or not the progeny is morphologically or genetically identical to the original parental cell, as long as the selected target gene is present in the progeny. Commonly used host cells include bacteria, yeast, mammalian cells, etc.

The term “vector” refers to a nucleic acid molecule capable of proliferating another nucleic acid to which it is linked. The term includes vectors that are self-replicating nucleic acid structures and vectors that are incorporated into the genome of the host cell into which they are introduced. Some vectors are capable of directing the expression of the nucleic acid to which they are operably linked.

“ADCC” refers to Antibody-Dependent Cell-mediated Cytotoxicity, where the Fab segment of an antibody binds to antigenic epitopes of virally infected cells or tumor cells. Its Fc segment binds to the FcR on the surface of killer cells (NK cells, macrophages, neutrophils, etc.) and mediates the direct killing of target cells by the killer cells, which is an action-important mechanism by which anti-tumor therapeutic antibody drugs act.

“ADCP” refers to Antibody-Dependent Cellular Phagocytosis, an important mechanism used to identify and mediate the action of therapeutic antibody with tumor cells.

### BRIEF DESCRIPTION OF THE DRAWINGS

FIGS. 1a-1d show schematic structures of bispecific antigen binding proteins. wherein the IgG portion in the schematic of FIG. 1a is an anti-4-1BB antibody (anti-4-1BB IgG), and the light and heavy chain variable regions of the anti-CD24 antibody are linked through a linker L1 to form scFv (VLCD24-L1-VHCD24 or VHCD24-L1-VLCD24), and the anti-CD24 scFv portion is linked by a linker L2 to the N-terminus of the anti-4-1BB IgG light chain; the IgG portion in the schematic of FIG. 1b is an anti-4-1BB antibody (anti-4-1BB IgG), and the light and heavy chain variable regions of the anti-CD24 antibody are linked through a linker L1 to form scFv (VLCD24-L1-VHCD24 or VHCD24-L1-VLCD24), and the anti-CD24 scFv portion is linked by a linker L2 to the N-terminus of the anti-4-1BB IgG heavy chain; the IgG portion in the schematic of FIG. 1c is an anti-CD24 antibody (anti-CD24 IgG), and the light and heavy chain variable regions of the anti-4-1BB antibody are linked through a linker L1 to form scFv (VL4-1BB-L1-VH4-1BB or VH4-1BB-L1-VL4-1BB), and the anti-4-1BB scFv portion is linked by a linker L2 to the C-terminus of the anti-CD24 IgG heavy chain; the IgG portion in the schematic of FIG. 1d is an anti-CD24 antibody (anti-CD24 IgG), and the light and heavy chain variable regions of the anti-4-1BB antibody are linked through a linker L1 to form scFv (VL4-1BB-L1-VH4-1BB or VH4-1BB-L1-VL4-1BB), and the anti-4-1BB scFv portion is linked by a linker L2 to the C-terminus of the anti-CD24 IgG light chain.

FIG. 2a-FIG. 2c show a schematic illustration of the binding activity of bispecific antigen binding proteins to CHOK1-4-1BB cells.

FIGS. 3a-3b show a schematic illustration of the binding activity of bispecific antigen binding proteins to CD24-MDA-MB-231 cells.

FIG. 4 shows a schematic illustration of the binding activity of bispecific antigen binding proteins to MCF-7 cells.

FIG. 5 shows a schematic illustration of the activation of bispecific antigen binding proteins on HEK-293/NFκB-Luci/4-1BB cells in the presence of target cells CHOK1-CD24.

FIGS. 6a-6b show a schematic illustration of the activation of bispecific antigen binding proteins on HEK-293/NFκB-Luci/4-1BB cells in the presence of target cell CD24-MDA-MB-231.

FIG. 7 shows a schematic illustration of the activation of bispecific antigen binding proteins on HEK-293/NFκB-Luci/4-1BB cells in the presence of target SKOV3.

FIG. 8 shows a schematic illustration of the activation of bispecific antigen binding proteins on HEK-293/NFκB-Luci/4-1BB cells in the presence of MCF-7 in target cells.

FIG. 9a shows a schematic illustration of the results of cytokine IFNγ release from PBMC pre-packed with PBS.

FIG. 9b shows a schematic illustration of the results of cytokine IFNγ release from PBMC preactivated with anti-human CD3 antibody.

FIG. 10 shows a schematic illustration of the results of the killing assay of SKOV3 cells by bispecific antigen binding proteins.

FIGS. 11a-11b show a schematic illustration of the ADCC effect of bispecific antigen binding proteins.

FIGS. 12a-12b show a schematic illustration of the ADCP effect of bispecific antigen binding proteins.

FIG. 13 shows a schematic illustration of the in vivo efficacy results of bispecific antigen binding proteins.

### DETAILED DESCRIPTION

In embodiments of the present invention, the heavy chain of CD24 monoclonal antibody-1 is shown in SEQ ID NO: 48 and the light chain is shown in SEQ ID NO: 49; The heavy chain of CD24 monoclonal antibody-2 is shown in SEQ ID NO: 50 and the light chain is shown in SEQ ID NO: 51; the heavy chain of 4-1BB monoclonal antibody is shown in SEQ ID NO: 52 and the light chain is shown in SEQ ID NO: 53. The IgG1 isotype control (Sino biological) and IgG4 isotype control (Keytruda Merck) were purchased commercially.

### Example 1 Preparation of Bispecific Antigen Binding Proteins with Different Structures

Sequence 1 of the present invention is a CD24 monoclonal antibody-1 sequence, Sequence 2 is a CD24 monoclonal antibody-2 sequence, and Sequence 3 is an anti-4-1BB antibody sequence. The amino acid sequences of the CDR and variable regions of sequence 1, sequence 2 and sequence 3, respectively, are shown in Table 1.

TABLE 1

| | Sequence 1 | Sequence 2 | Sequence 3 |
|---|---|---|---|
| Heavy chain | SEQ ID NO: 1 | SEQ ID NO: 9 | SEQ ID NO: 17 |
| variable region | | | |
| HCDR1 | SEQ ID NO: 2 | SEQ ID NO: 10 | SEQ ID NO: 18 |
| HCDR2 | SEQ ID NO: 3 | SEQ ID NO: 11 | SEQ ID NO: 19 |
| HCDR3 | SEQ ID NO: 4 | SEQ ID NO: 12 | SEQ ID NO: 20 |
| Light chain | SEQ ID NO: 5 | SEQ ID NO: 13 | SEQ ID NO: 21 |
| variable region | | | |
| LCDR1 | SEQ ID NO: 6 | SEQ ID NO: 14 | SEQ ID NO: 22 |
| LCDR2 | SEQ ID NO: 7 | SEQ ID NO: 15 | SEQ ID NO: 23 |
| LCDR3 | SEQ ID NO: 8 | SEQ ID NO: 16 | SEQ ID NO: 24 |

The peptide chain in Table 2 can be formed from sequence 1 and sequence 3 linked by linkers L1 and L2 in a certain order. Wherein, the bispecific antigen binding protein has the same sequence structure of linker 1 (L1) and linker 2 (L2) used in this embodiment, which are 3 GGGGS repeats, i.e., GGGGSGGGGSGGGGS, respectively.

TABLE 2

| Peptide | | |
|---|---|---|
| chain | | Amino acid |
| name | Linking order | sequence |
| DBH1 | VH_{4-1BB}-Fc4 | SEQ ID NO: 25 |
| DBH2 | VH_{CD24}-Fc4 | SEQ ID NO: 26 |
| DBH3 | VL_{CD24}-L1-VH_{CD24}-L2-VH_{4-1BB}-Fc4 | SEQ ID NO: 27 |
| DBH4 | VH_{CD24}-L1-VL_{CD24}-L2-VH_{4-1BB}-Fc4 | SEQ ID NO: 28 |
| DBH5 | VH_{CD24}-Fc4-L2-VL_{4-1BB}-L1-VH_{4-1BB} | SEQ ID NO: 29 |
| DBH6 | VH_{CD24}-Fc4-L2-VH_{4-1BB}-L1-VL_{4-1BB} | SEQ ID NO: 30 |
| DBH7 | VH_{CD24}-Fc1-L2-VL_{4-1BB}-L1-VH_{4-1BB} | SEQ ID NO: 31 |
| DBH8 | VH_{CD24}-Fc1-L2-VH_{4-1BB}-L1-VL_{4-1BB} | SEQ ID NO: 32 |
| DBL1 | VL_{4-1BB}-CL | SEQ ID NO: 33 |
| DBL2 | VL_{CD24}-CL | SEQ ID NO: 34 |
| DBL3 | VL_{CD24}-L1-VH_{CD24}-L2-VL_{4-1BB}-CL | SEQ ID NO: 35 |
| DBL4 | VH_{CD24}-L1-VL_{CD24}-L2-VL_{4-1BB}-CL | SEQ ID NO: 36 |
| DBL5 | VL_{CD24}-CL-L2-VL_{4-1BB}-L1-VH_{4-1BB} | SEQ ID NO: 37 |
| DBL6 | VL_{CD24}-CL-L2-VH_{4-1BB}-L1-VL_{4-1BB} | SEQ ID NO: 38 |

The peptide chain in Table 3 can be formed from sequence 2 and sequence 3 linked by linkers L1 and L2 in a certain order.

TABLE 3

| Peptide | | |
|---|---|---|
| chain | | Amino acid |
| name | Linking order | sequence |
| H1 | VH_{CD24}-L1-VL_{CD24}-L2-VH_{4-1BB}-Fc1 | SEQ ID NO: 39 |
| H2 | VH_{CD24}-L1-VL_{CD24}-L2-VH_{4-1BB}-Fc4 | SEQ ID NO: 40 |
| H3 | VH_{CD24}-Fc1-L2-VH_{4-1BB}-L1-VL_{4-1BB} | SEQ ID NO: 41 |
| H4 | VH_{CD24}-Fc4-L2-VH_{4-1BB}-L1-VL_{4-1BB} | SEQ ID NO: 42 |
| H5 | VH_{CD24}-Fc4 | SEQ ID NO: 43 |
| H6 | VH_{CD24}-Fc1 | SEQ ID NO: 44 |
| L1 | VL_{4-1BB}-CL | SEQ ID NO: 45 |
| L2 | VL_{CD24}-CL | SEQ ID NO: 46 |
| L3 | VL_{CD24}-CL-L2-VH_{4-1BB}-L1-VL_{4-1BB} | SEQ ID NO: 47 |

From the combination of peptide chains in Tables 2 and 3, the bispecific antigen binding proteins shown in Table 4 can be formed.

TABLE 4

| Structural names of | Heavy chain | Light chain |
|---|---|---|
| antigen binding | amino acid | amino acid |
| proteins | sequence | sequence |
| 1-A1-IgG4 | SEQ ID NO: 25 | SEQ ID NO: 35 |
| 1-A2-IgG4 | SEQ ID NO: 25 | SEQ ID NO: 36 |
| 1-B1-IgG4 | SEQ ID NO: 27 | SEQ ID NO: 33 |
| 1-B2-IgG4 | SEQ ID NO: 28 | SEQ ID NO: 33 |
| 1-C1-IgG4 | SEQ ID NO: 29 | SEQ ID NO: 34 |
| 1-C2-IgG4 | SEQ ID NO: 30 | SEQ ID NO: 34 |
| 1-C1-IgG1 | SEQ ID NO: 31 | SEQ ID NO: 34 |
| 1-C2-IgG1 | SEQ ID NO: 32 | SEQ ID NO: 34 |
| 1-D1-IgG4 | SEQ ID NO: 26 | SEQ ID NO: 37 |
| 1-D2-IgG4 | SEQ ID NO: 26 | SEQ ID NO: 38 |
| 2-B2-IgG1 | SEQ ID NO: 39 | SEQ ID NO: 45 |
| 2-B2-IgG4 | SEQ ID NO: 40 | SEQ ID NO: 45 |
| 2-C2-IgG1 | SEQ ID NO: 41 | SEQ ID NO: 46 |
| 2-C2-IgG4 | SEQ ID NO: 42 | SEQ ID NO: 46 |
| 2-D2-IgG1 | SEQ ID NO: 44 | SEQ ID NO: 47 |
| 2-D2-IgG4 | SEQ ID NO: 43 | SEQ ID NO: 47 |

The above-designed different structures of each chain of bispecific antigen binding proteins were genetically synthesized and constructed into the human IgG framework, and then the antibody fragments were inserted into PCDNA3.1 vector using molecular cloning technology, constructed into a mammalian cell expression plasmid, and then imported into CHO cells of the host cell line using liposome transfection, and the fermentation supernatant was obtained by using cell fed-batch. The fermentation supernatant was taken for purification by affinity chromatography, ion exchange chromatography and a series of other steps, and the constructed antibody was finally purified. The purified antibody was tested for expression amount, purity, SDS-PAGE, etc. to confirm the the bispecific antigen binding protein.

### Example 2 Binding Activity of Bispecific Antigen Binding Proteins to CHOK1-4-1BB, CD24-MDA-MB-231, and MCF-7

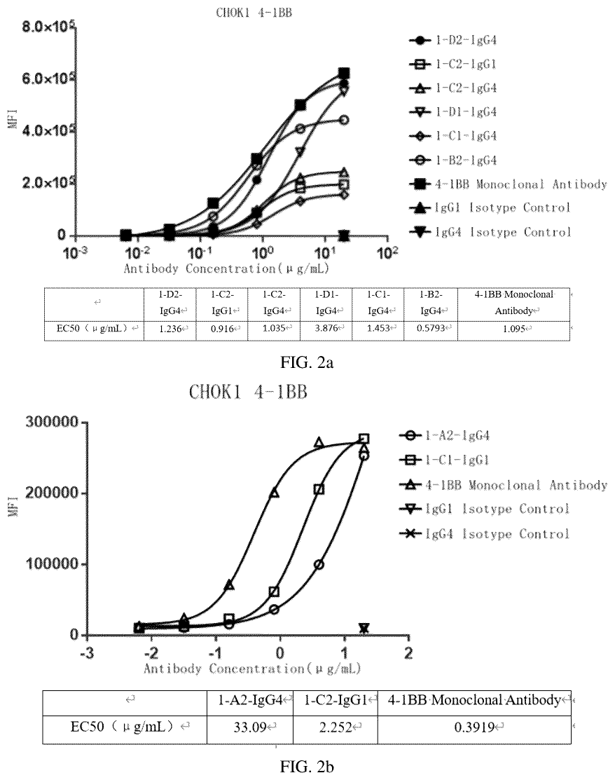

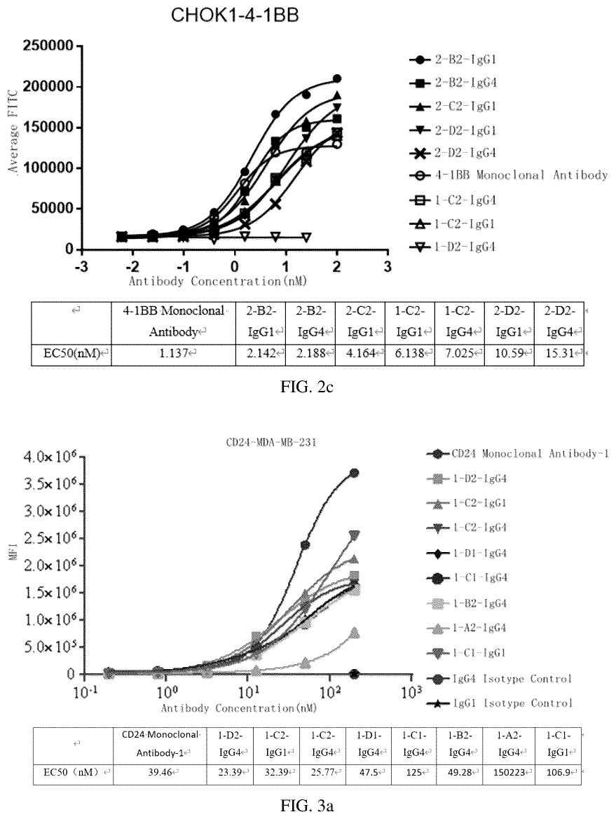

A lentiviral vector carrying human 4-1BB was transduced into the CHOK1 cell line (ATCC), and the cells were sorted to establish the human 4-1BB stably expressing cell line CHOK1-4-1BB. A lentiviral vector carrying human CD24 was transduced into the MDA-MB-231 breast cancer cell line (ATCC), and the cells were sorted to establish the human CD24 stably expressing cell line CD24-MDA-MB-231. The natural tumor cell line MCF-7 was derived from ATCC (American Model Culture Collection) and highly expresses CD24. The various types of cells were taken and transferred to a centrifuge tube and centrifuged at 1000 rpm for 5 minutes. Separately, 100 μL of each cell was spread in a 96U plate and washed twice with PBS. Bispecific antigen binding proteins of different structures were added to the 9 6U-type plates spread with tumor cells starting at a final concentration of 200 nM, with 4-fold dilution in 6 gradients with 100 μL/well. Cells were incubated at 37° C. for 60 min and then washed twice with excess FACS buffer. Cells were resuspended in 100 μL of FACS buffer with fluorescent secondary antibody (FITC F(ab′)₂ Sheep Anti-Human IgG Fcγ Antibody) added to the samples, and incubated for 30 min at 37° C. and washed twice with excess FACS buffer. The cells were fixed in fixation buffer and subsequently analyzed by flow cytometry. The experimental results are shown in FIGS. 2a-2c, 3a-3b and 4.

As shown from FIGS. 2a-2c, the constructed bispecific antigen binding proteins with different structures show very good binding activities with CHOK1-4-1BB cells, with EC₅₀ reaching 0.5793-33.09 g/mL or 2.188-15.31 nM, and the binding activities are all significantly better than those of the IgG1 isotype control and the IgG4 isotype control.

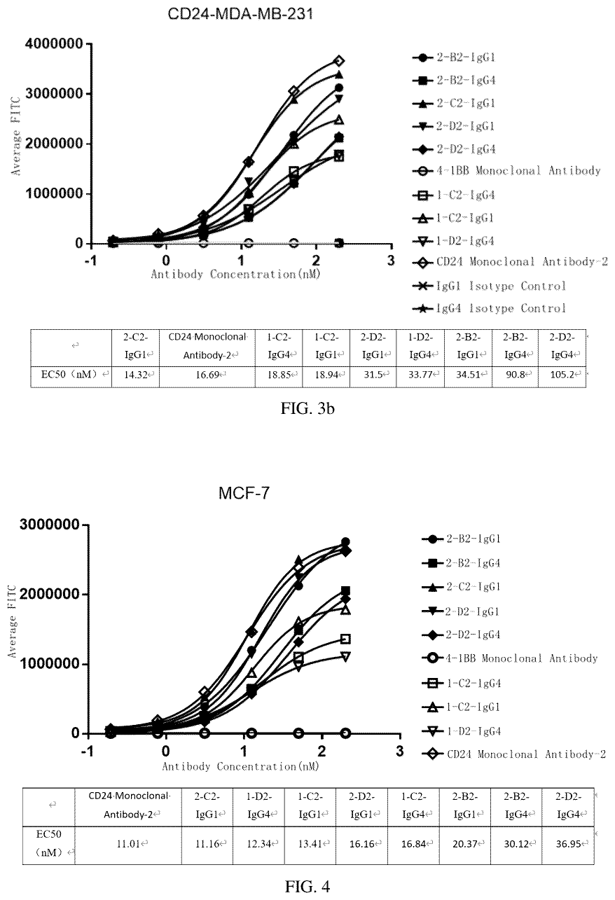

As shown from FIGS. 3a-3b, the constructed bispecific antigen binding proteins with different structures show very good binding activities with CD24-MDA-MB-231 cells, with EC₅₀ reaching 14.32-106.9 nM, and the binding activities are all significantly better than those of the IgG1 isotype control and the IgG4 isotype control.

As shown from FIG. 4, the constructed bispecific antigen binding proteins with different structures show very good binding activities with MCF-7 cells, with EC₅₀ reaching 11.16-36.95 nM, which was significantly better than that of 4-1BB monoclonal antibody.

### Example 3 Activation of HEK-293/NFκB-Luci/4-1BB Cells by Bispecific Antigen Binding Proteins

The cell fluorescence values were detected by enzyme marker, which reflected the activation of different structures of bispecific antigen binding proteins on HEK-293/NFκB-Luci/4-1BB cells in the presence of different target cells (CHOK1-CD24, CD24-MDA-MB-231, MCF-7, SKOV3). Wherein, SKOV3 was obtained from the Cell Bank of the Chinese Academy of Sciences.

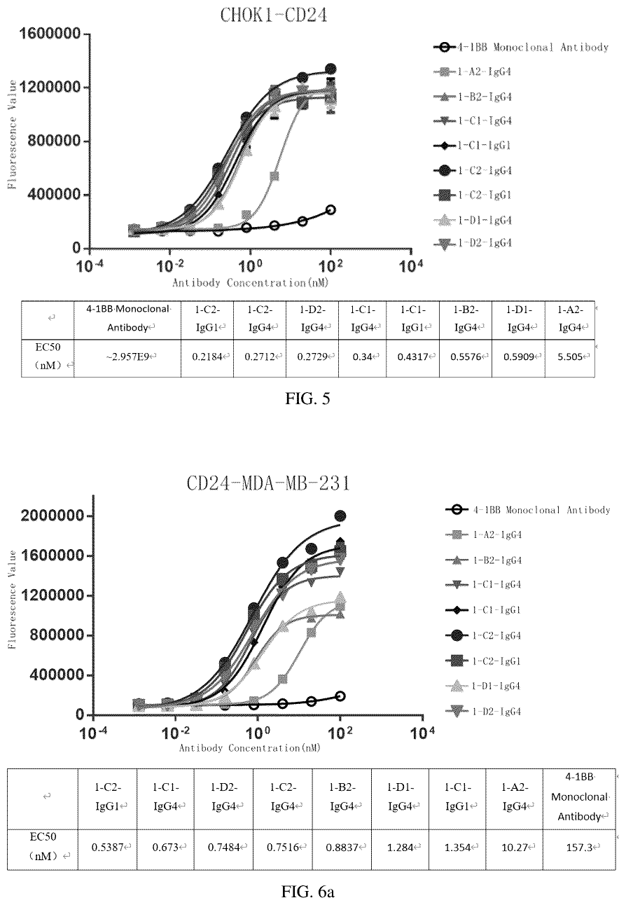

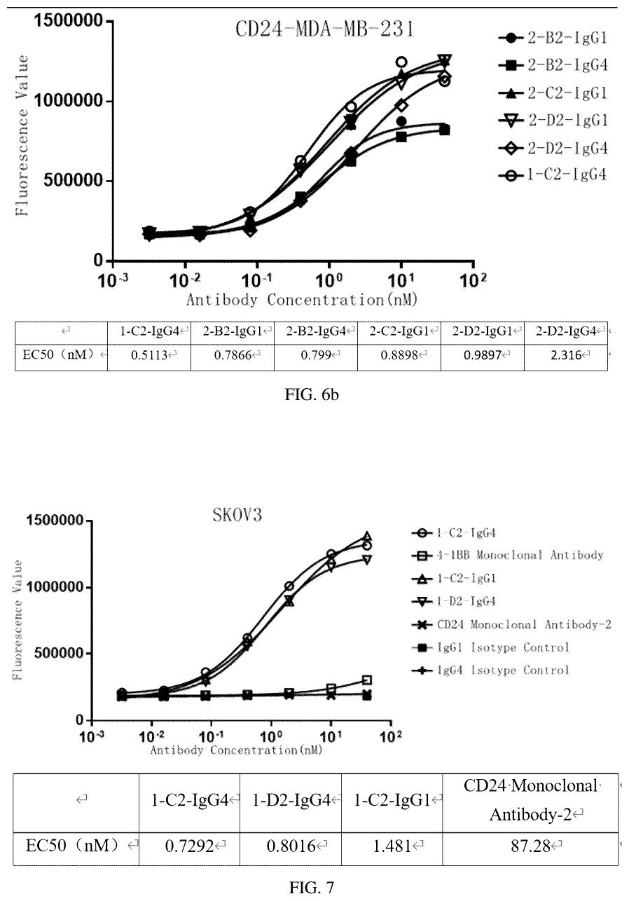

HEK-293/NFκB-Luci/4-1BB cells (purchased from Hankook Mab) (3×10⁴ cells/well, 40 μL/well) were spread in a 96-well plate, and then 40 μL of each target cell was added, with 1×10⁴ cells/well. Then the antibody for each structure was added separately, and the final concentration of antibody was 200 nM starting, 5-fold dilution in 8 gradients with 20 μL/well. The 96-well plate was incubated in a 37° C. incubator for 18 h, and finally the color developer was added to develop the color, and the fluorescence value was detected at 570 nm. The experimental results are shown in FIG. 5, FIGS. 6a-6b, FIG. 7 and FIG. 8.

As shown from FIG. 5, bispecific antigen binding proteins in the presence of target cell CHOK1-CD24 were well activated in HEK-293/NFκB-Luci/4-1BB cells, with EC₅₀ of 0.2184-5.505 nM, with no activation by 4-1BB monoclonal antibody.

As shown from FIGS. 6a-6b, bispecific antigen binding proteins in the presence of target cell CD24-MDA-MB-231 were well activated in HEK-293/NFκB-Luci/4-1BB cells, with EC₅₀ of 0.5113-10.27 nM, with no activation by 4-1BB monoclonal antibody.

As shown from FIG. 7, bispecific antigen binding proteins in the presence of target cell SKOV3 were well activated in HEK-293/NFκB-Luci/4-1BB cells, with EC₅₀ of 0.7292-1.481 nM, with no activation by 4-1BB monoclonal antibody.

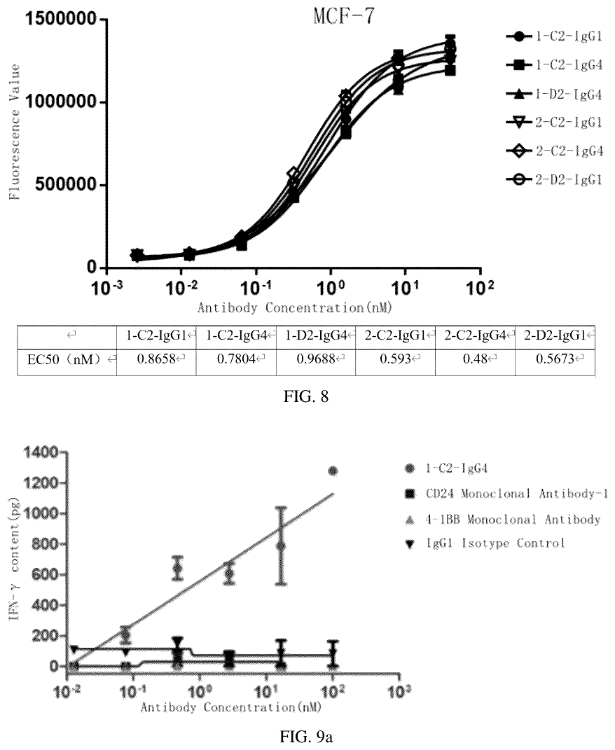

As shown from FIG. 8, bispecific antigen binding proteins in the presence of target cell MCF-7 were well activated in HEK-293/NFκB-Luci/4-1BB cells, with EC₅₀ of 0.48-0.9688 nM, with no activation by 4-1BB monoclonal antibody.

It can be shown that bispecific antigen binding proteins have a strong activating activity on the 4-1BB signaling pathway through cross-linking of the CD24 terminus in the presence of target cells CHOK1-CD24, CD24-MDA-MB-231, MCF-7, or SKOV3.

### Example 4 the Ability of Bispecific Antigen Binding Proteins to Activate Cytokine Release from PBMC and their Killing Effect on Tumor Cells

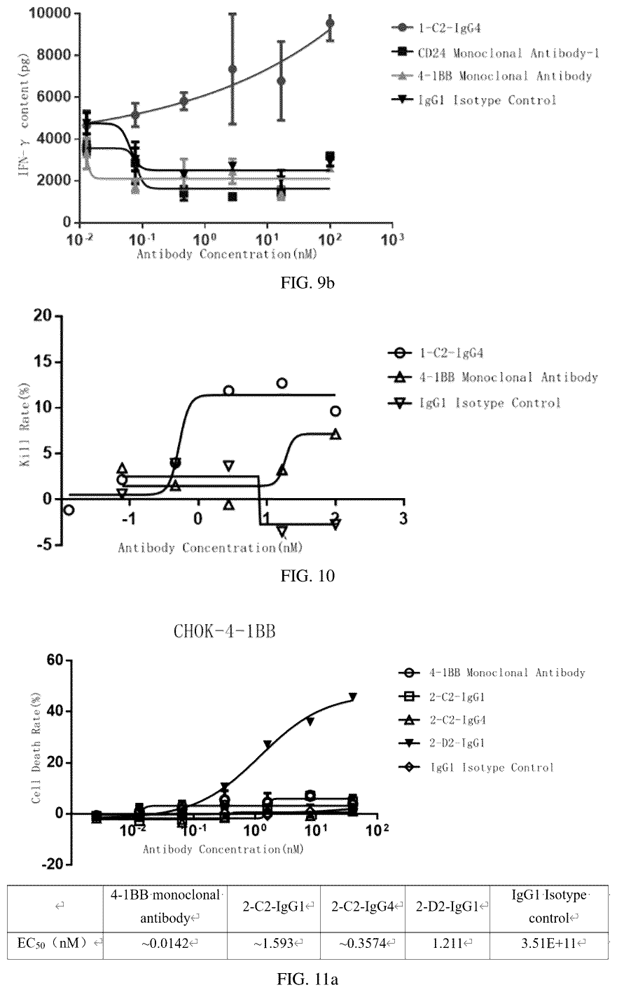

PBMC at 1×10⁶ cells/mL were preactivated with either 5 g/mL of anti-human CD3 antibody (BioGems; FPB004-C) or an equal amount of PBS for 24 h at 37° C. The target cells SKOV3 were plated at 5000/100 μL/well, and after overnight incubation at 37° C., the target cell plate medium was discarded and replaced with 1640+10% FBS with 50 μL/well. Then 50 μL/well of each antibody of different structures were added, and the antibody was 6-fold diluted according to the final concentration starting at 100 nM. At the same time, pre-activated PBMC were added according to the potency-target ratio of 5:1. Supernatants were taken after incubation at 37° C. for 24 h, and the release of IFN-γ was detected by ELISA using a kit (R&D Systems, SIF50). The remaining cells were added to CCK8 developing solution at 20 μL/well, and the target cell survival rate was analyzed by detecting the OD value at 490 nm with an enzyme marker after incubation for 4 h at 37° C. The experimental results are shown in FIGS. 9a-9b and FIG. 10. Wherein, FIG. 9a shows the results of cytokine IFNγ release from PBMC pre-packed with PBS, and FIG. 9b shows the results of cytokine IFNγ release from PBMC pre-activated with anti-human CD3 antibody.

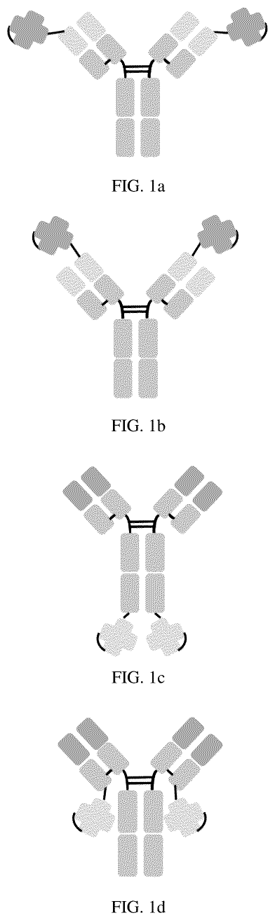

As shown from FIGS. 9a-9b, 1-C2-IgG4 activated PBMC-releasing cytokine IFNγ pre-activated with PBS pre-packed plates or anti-human CD3 antibody in a dose-dependent manner compared to IgG1 isotype control. CD24 monoclonal antibody-1 and 4-1BB monoclonal antibody failed to activate PBMC-releasing cytokine IFNγ at any dose.

As shown from FIG. 10, 1-C2-IgG4 shows the ability to kill SKOV3 cells at a rate greater than 10%, while 4-1BB monoclonal antibody shows weak killing of SKOV3 cells only at high concentrations, indicating that the killing of target cells by 1-C2-IgG4 is a target cell-dependent and specific killing.

### Example 5 ADCC Activity of Bispecific Antigen Binding Proteins on Target Cells

CHOK1-4-1BB or MCF-7 cells were used, and after centrifugation at 1000 rpm for 4 min at room temperature and resuspension with RPMI1640 basal medium (containing 5% FBS), were spread in a 96-well plate at 1×10⁴/well with 50 μL/well; the constructed double antibody was diluted to 50 nM using RPMI1640 basal medium (containing 5% FBS), while it was later diluted in a 5-fold gradient, with a total of 7 concentration gradients and 100 μL/well; NK cells were resuspended and added to the corresponding wells with 50 μL/well, in which the target cell was CHOK1-4-1BB with a 3:1 time-target ratio, and the target cell was MCF-7 with a 5:1 time-target ratio. Target cell maximum lysis wells (M), target cell spontaneous release wells (ST), effector cell spontaneous release wells (SE), total volume-corrected blank wells (BV), and medium blank control wells (BM) were set up at the same time. After standing for 10 min, centrifugation was performed at 1000 rpm for 4 min at room temperature and incubated for 4 h in a 5% C02, and 37° C. carbon dioxide cell culture incubator. Lysate was added to wells M and B-V 45 min in advance, mixed well, and centrifuged at 1000 rpm for 4 min at room temperature at the end of incubation. 50 μL of supernatant was aspirated to the LDH analysis plate, 50 μL/well of substrate dissolved in analysis buffer (assay buffer) was added, and the reaction was performed for 30 min at room temperature and protected from light. 50 μL/well of termination solution was added and stood for 10 min, readings were taken at 490 nm and cell death rate was calculated.

$$Cell Death Rate ( \% ) = \frac{{ODvalue}_{cuvette - ST - SE} \times 100}{{ODvalue}_{M - ST}} .$$

As shown from FIG. 11a, IgG1 isotype control and IgG4 isotype control do not show killing of CHOK-4-1BB cells, and 2-D2-IgG1 shows lysogenic death of CHOK-4-1BB cells in a concentration-dependent manner with an EC₅₀ of 1.211 nM.

As shown from FIG. 11b, IgG1 isotype control and IgG4 isotype control do not show killing of MCF-7 cells, and both 2-C2-IgG1 and 2-D2-IgG1 show cleavage death of MCF-7 cells in a concentration-dependent manner, with EC₅₀ of 0.1516 nM and 0.4143 nM, respectively.

### Example 6 ADCP Effect of Bispecific Antigen Binding Proteins

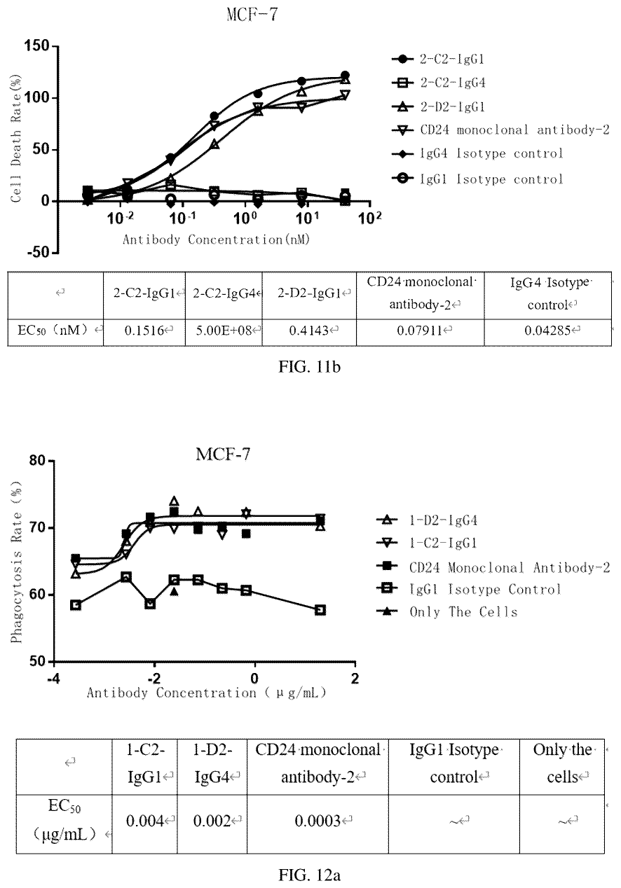

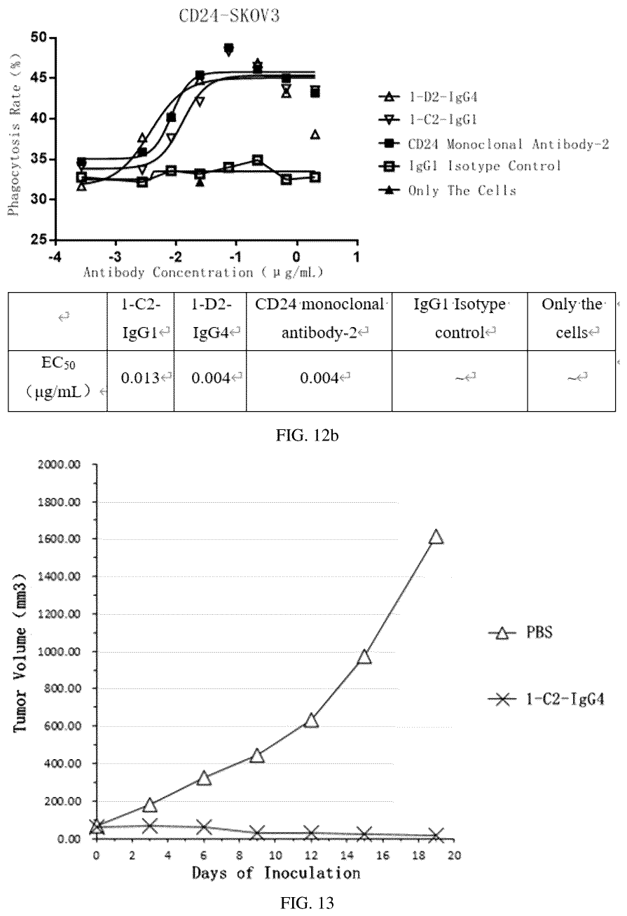

Monocytes were cultured in RPMI1640 medium containing 50 ng/mL rhM-CSF (Kactus, CSF-HM401) for 10 days. It was centrifuged, and MCF-7 cells (or CD24-SKOV3 cells) were washed with PBS. Then it was stained with CFSE, and incubated at 30° C. for 20 minutes. 50 μL of MCF-7 cells were added to the plate, and 50 μL of bispecific antigen binding protein dilution was added to the cells. 100 μL of macrophage suspension was added to the cells and incubated at 37° C. for 3 hours. The above cells were centrifuged and the supernatant discarded, and 100 μL of APC-labeled anti-human CD14 (eBioscience™, 17-0149-42) was added to the cells and incubated at 4° C. for 20 min. The cells were centrifuged and washed twice with FACS buffer and resuspended with FACS buffer. The results were tested by flow cytometry and the results are shown in FIGS. 12a-12b.

As shown from FIG. 12a, both 1-C2-IgG1 and 1-D2-IgG4 significantly induced phagocytosis of MCF-7 cells by macrophages with EC₅₀ of 0.004 nM and 0.002 nM, respectively, and the phagocytosis was much stronger than that of the IgG1 isotype control.

As shown from FIG. 12a, both 1-C2-IgG1 and 1-D2-IgG4 significantly induced phagocytosis of CD24-SKOV3 cells by macrophages with EC₅₀ of 0.013 nM and 0.004 nM, respectively, and the phagocytosis was much stronger than that of the IgG1 isotype control.

### Example 7 In Vivo Efficacy of Bispecific Antigen Binding Proteins in CD24-MC38 Transplanted Tumor

The h4-1BB transgenic mouse (purchased from Shanghai Southern Model Biotechnology Co., Ltd.) colorectal cancer CD24-MC38 cell subcutaneous tumor model was constructed by the colorectal cancer cell line CD24-MC38 (Jiman Bio, GM-C15769), which stably expresses human CD24. Tumor cells were resuscitated, cell culture was performed and cell status was adjusted to optimal, and cell suspension was made after digestion. When the cells were cultured to the logarithmic growth phase, the cells were collected, and the tumor cell suspension was injected subcutaneously into the mouse, each inoculated with 100 μL of cell suspension containing 5×10⁶ cells. When the subcutaneously grown tumors grew to about 100 mm³, the animals were randomly grouped according to tumor volume and administered bispecific antigen binding protein.

Bispecific antigen binding protein and PBS were administered intraperitoneally to grouped homozygous mouse at 10.0 mg/kg twice a week for 5 doses, respectively.

The tumor suppression efficacy of the compounds was evaluated by TGI (%). Formula of TGI (%): TGI (%)=[(1−(average tumor volume at the end of the administration of a treatment group−average tumor volume at the beginning of the administration of that treatment group))/(average tumor volume at the end of the treatment of the solvent control group− average tumor volume at the beginning of the treatment of the solvent control group)]×100%.

The tumor growth curves of mouse colorectal cancer subcutaneous tumor model of hormonal mouse given bispecific antigen binding proteins are shown in FIG. 13, where the horizontal coordinate indicates the number of days after the start of treatment and the vertical coordinate indicates the tumor volume. The tumor suppression rate TGI (%) was 100%.

As shown from FIG. 13, 1-C2-IgG4 has a good tumor inhibitory effect and a very significant decrease in tumor volume.

The scope of protection of the present invention is not limited to the above embodiments. Without departing from the spirit and scope of the inventive conception, variations and advantages that can be thought of by those skilled in the art are included in the present invention and are protected by the appended claims.

1. A bispecific antigen binding protein, comprising:
(a) a first antibody or antigen binding fragment thereof that specifically binds to a first antigen; and
(b) a second antibody or antigen binding fragment thereof that specifically binds to a second antigen;
wherein
the first antigen is CD24, and the second antigen is 4-1BB; or
the first antigen is 4-1BB, and the second antigen is CD24.

2. The bispecific antigen binding protein of claim 1, wherein the first antibody or antigen binding fragment thereof comprises a heavy chain and a light chain;
the second antibody or antigen binding fragment thereof comprises a scFv; and
the scFv is linked to the N-terminal or C-terminal end of the heavy or light chain of the first antibody or antigen binding fragment thereof.

3. The bispecific antigen binding protein of claim 1, wherein the heavy chain variable region of one heavy chain of the first antibody or antigen binding fragment thereof forms an antigen binding site with the light chain variable region of one light chain, and the heavy chain variable region of another heavy chain forms an antigen binding site with the light chain variable region of another light chain.

4. The bispecific antigen binding protein of claim 3, further comprising one first antibody or antigen binding fragment thereof and one or more scFv(s).

5. The bispecific antigen binding protein of claim 4, further comprising one first antibody or antigen binding fragment thereof and two scFvs.

6. The bispecific antigen binding protein of claim 5, wherein one scFv is linked to the N-terminal end of the heavy or light chain of the first antibody or antigen binding fragment thereof, and the other scFv is linked to the C-terminal end of the heavy or light chain of the first antibody or antigen binding fragment thereof.

7. The bispecific antigen binding protein of claim 5, wherein two scFvs are linked to the N-terminal ends of two heavy chains or two light chains of the first antibody or the antigen binding fragment thereof, respectively; or two scFvs are linked to the C-terminal ends of two heavy chains or two light chains of the first antibody or the antigen binding fragment thereof, respectively.

8. The bispecific antigen binding protein of claim 7, further comprising two first polypeptide chains and two second polypeptide chains, and, for each of the polypeptide chains:
(a) the first polypeptide chain each independently comprises the heavy chain of the first antibody or antigen binding fragment thereof and the scFv; and
(b) the second polypeptide chain each independently comprises the light chain of the first antibody or antigen binding fragment thereof.

9. The bispecific antigen binding protein of claim 7, further comprising two first polypeptide chains and two second polypeptide chains, and, for each of the polypeptide chains:
(a) the first polypeptide chain each independently comprises the heavy chain of the first antibody or antigen binding fragment thereof, and
(b) the second polypeptide chain each independently comprises the light chain of the first antibody or antigen binding fragment thereof and the scFv.

10. The bispecific antigen binding protein of claim 9, wherein two first polypeptide chains are the same or different, and/or two second polypeptide chains are the same or different.

11. The bispecific antigen binding protein of claim 10, wherein the heavy chain variable region of the scFv is linked to the light chain variable region by a linker L1, and/or two scFvs are linked to the N-terminal or C-terminal ends of two heavy chains or two light chains of the first antibody or antigen binding fragment thereof, respectively, by a linker L2.

12. The bispecific antigen binding protein of claim 11, wherein the linker L1 and the linker L2 are the same or different.

13. The bispecific antigen binding protein of claim 12, wherein the linker L1 and/or the linker L2 have an amino acid sequence as shown in (G₄S)ₓ, x is an integer selected from 1-6; preferably, the linker L1 and/or L2 is (G₄S)₃ or (G₄S)₄.

14. The bispecific antigen binding protein of claim 13, wherein a disulfide bond exists between the heavy chain variable region and the light chain variable region of the scFv.

15. The bispecific antigen binding protein of claim 14, wherein the heavy chain of the first antibody or antigen binding fragment thereof comprises a heavy chain variable region and a heavy chain constant region, and the light chain comprises a light chain variable region and a light chain constant region.

16-19. (canceled)

20. The bispecific antigen binding protein of claim 19, wherein the first antibody or antigen binding fragment thereof specifically binds to CD24, and the scFv specifically binds to 4-1BB, wherein,
the first antibody or antigen binding fragment thereof comprises:
(a) HCDR1 as shown in SEQ ID NO: 2, HCDR2 as shown in SEQ ID NO: 3, HCDR3 as shown in SEQ ID NO: 4; and, LCDR1 as shown in SEQ ID NO: 6, LCDR2 as shown in SEQ ID NO: 7, and LCDR3 as shown in SEQ ID NO: 8; or
(b) HCDR1 as shown in SEQ ID NO: 10, HCDR2 as shown in SEQ ID NO: 11, and HCDR3 as shown in SEQ ID NO: 12; and, LCDR1 as shown in SEQ ID NO: 14, LCDR2 as shown in SEQ ID NO: 15, and LCDR3 as shown in SEQ ID NO: 16;
and the scFv comprises:
HCDR1 as shown in SEQ ID NO: 18, HCDR2 as shown in SEQ ID NO: 19, and HCDR3 as shown in SEQ ID NO: 20; and, LCDR1 as shown in SEQ ID NO: 22, LCDR2 as shown in SEQ ID NO: 23, and LCDR3 as shown in SEQ ID NO: 24.

21. The bispecific antigen binding protein of claim 1, wherein the first antibody or antigen binding fragment thereof comprises:
(a) a heavy chain variable region VH as shown in SEQ ID NO: 1, and a light chain variable region VL as shown in SEQ ID NO: 5; or
(b) a heavy chain variable region VH as shown in SEQ ID NO: 9, and a light chain variable region VL as shown in SEQ ID NO: 13;
and the scFv comprises:
a heavy chain variable region VH as shown in SEQ ID NO: 17, and a light chain variable region VL as shown in SEQ ID NO: 21.

22. The bispecific antigen binding protein of claim 1, wherein the first antibody or antigen binding fragment thereof specifically binds to 4-1BB, and the scFv specifically binds to CD24, wherein,
the first antibody or antigen binding fragment thereof comprises:
HCDR1 as shown in SEQ ID NO: 18, HCDR2 as shown in SEQ ID NO: 19, HCDR3 as shown in SEQ ID NO: 20; and, LCDR1 as shown in SEQ ID NO: 22, LCDR2 as shown in SEQ ID NO: 23, LCDR3 as shown in SEQ ID NO: 24;
and the scFv comprises:
(a) HCDR1 as shown in SEQ ID NO: 2, HCDR2 as shown in SEQ ID NO: 3, HCDR3 as shown in SEQ ID NO: 4; and, LCDR1 as shown in SEQ ID NO: 6, LCDR2 as shown in SEQ ID NO: 7, and LCDR3 as shown in SEQ ID NO: 8; or
(b) HCDR1 as shown in SEQ ID NO: 10, HCDR2 as shown in SEQ ID NO: 11, HCDR3 as shown in SEQ ID NO: 12; and, LCDR1 as shown in SEQ ID NO: 14, LCDR2 as shown in SEQ ID NO: 15, and LCDR3 as shown in SEQ ID NO: 16.

23. The bispecific antigen binding protein of claim 1, wherein the first antibody or antigen binding fragment thereof comprises:
a heavy chain variable region VH as shown in SEQ ID NO: 17, and a light chain variable region VL as shown in SEQ ID NO: 21;
and the scFv comprises:
(a) a heavy chain variable region VH as shown in SEQ ID NO: 1, and a light chain variable region VL as shown in SEQ ID NO: 5; or
(b) a heavy chain variable region VH as shown in SEQ ID NO: 9, and a light chain variable region VL as shown in SEQ ID NO: 13.

24. The bispecific antigen binding protein of claim 1, wherein the bispecific antigen binding protein comprises:
(1) a first polypeptide chain as shown in SEQ ID NO: 25, and a second polypeptide chain as shown in SEQ ID NO: 35;
(2) a first polypeptide chain as shown in SEQ ID NO: 25, and a second polypeptide chain as shown in SEQ ID NO: 36;
(3) a first polypeptide chain as shown in SEQ ID NO: 27, and a second polypeptide chain as shown in SEQ ID NO: 33;
(4) a first polypeptide chain as shown in SEQ ID NO: 28, and a second polypeptide chain as shown in SEQ ID NO: 33;
(5) a first polypeptide chain as shown in SEQ ID NO: 29, and a second polypeptide chain as shown in SEQ ID NO: 34;
(6) a first polypeptide chain as shown in SEQ ID NO: 30, and a second polypeptide chain as shown in SEQ ID NO: 34;
(7) a first polypeptide chain as shown in SEQ ID NO: 31, and a second polypeptide chain as shown in SEQ ID NO: 34;
(8) a first polypeptide chain as shown in SEQ ID NO: 32, and a second polypeptide chain as shown in SEQ ID NO: 34;
(9) a first polypeptide chain as shown in SEQ ID NO: 26, and a second polypeptide chain as shown in SEQ ID NO: 37;
(10) a first polypeptide chain as shown in SEQ ID NO: 26, and a second polypeptide chain as shown in SEQ ID NO: 38;
(11) a first polypeptide chain as shown in SEQ ID NO: 39, and a second polypeptide chain as shown in SEQ ID NO: 45;
(12) a first polypeptide chain as shown in SEQ ID NO: 40, and a second polypeptide chain as shown in SEQ ID NO: 45;
(13) a first polypeptide chain as shown in SEQ ID NO: 41, and a second polypeptide chain as shown in SEQ ID NO: 46;
(14) a first polypeptide chain as shown in SEQ ID NO: 42, and a second polypeptide chain as shown in SEQ ID NO: 46;
(15) a first polypeptide chain as shown in SEQ ID NO: 43, and a second polypeptide chain as shown in SEQ ID NO: 47; or
(16) a first polypeptide chain as shown in SEQ ID NO: 44, and a second polypeptide chain as shown in SEQ ID NO: 47.

25-29. (canceled)


SEQUENCE LISTING (53 sequences)

SEQ ID NO: 1 - protein, 112 aa, synthetic construct
QVQLVQSGSELKKPGASVKVSCKASGYIFTDYAIHWVKQAPGQGLKWMGFINTATGEPTYADDFKGRFVFSLDTSVSTAYLQISSLKAEDTAVYFCTSGVHWGQGTLVTVSS

SEQ ID NO: 2 - protein, 8 aa, synthetic construct
GYIFTDYA

SEQ ID NO: 3 - protein, 8 aa, synthetic construct
INTATGEP

SEQ ID NO: 4 - protein, 5 aa, synthetic construct
TSGVH

SEQ ID NO: 5 - protein, 106 aa, synthetic construct
DIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGGGTKVEIK

SEQ ID NO: 6 - protein, 10 aa, synthetic construct
SASSSVSSMH

SEQ ID NO: 7 - protein, 7 aa, synthetic construct
STSNLAS

SEQ ID NO: 8 - protein, 9 aa, synthetic construct
HHWTSYMYT

SEQ ID NO: 9 - protein, 117 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSS

SEQ ID NO: 10 - protein, 6 aa, synthetic construct
SGYTWH

SEQ ID NO: 11 - protein, 16 aa, synthetic construct
HIQYTGNTKYNPSLES

SEQ ID NO: 12 - protein, 8 aa, synthetic construct
GSTNSNDY

SEQ ID NO: 13 - protein, 113 aa, synthetic construct
DIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELK

SEQ ID NO: 14 - protein, 17 aa, synthetic construct
KSSQSLLYSNDQKNYLA

SEQ ID NO: 15 - protein, 7 aa, synthetic construct
WASTRES

SEQ ID NO: 16 - protein, 9 aa, synthetic construct
QQYFIYPLT

SEQ ID NO: 17 - protein, 117 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSS

SEQ ID NO: 18 - protein, 5 aa, synthetic construct
SYGVH

SEQ ID NO: 19 - protein, 16 aa, synthetic construct
VIWPGGSTNYNSALMS

SEQ ID NO: 20 - protein, 9 aa, synthetic construct
VTGTWYFDV

SEQ ID NO: 21 - protein, 107 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIK

SEQ ID NO: 22 - protein, 11 aa, synthetic construct
SASQGISNYLN

SEQ ID NO: 23 - protein, 7 aa, synthetic construct
YTSTLHS

SEQ ID NO: 24 - protein, 9 aa, synthetic construct
QQYSKLPWT

SEQ ID NO: 25 - protein, 444 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 439 aa, synthetic construct
QVQLVQSGSELKKPGASVKVSCKASGYIFTDYAIHWVKQAPGQGLKWMGFINTATGEPTYADDFKGRFVFSLDTSVSTAYLQISSLKAEDTAVYFCTSGVHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 692 aa, synthetic construct
DIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGCGTKVEIKGGGGSGGGGSGGGGSQVQLVQSGSELKKPGASVKVSCKASGYIFTDYAIHWVKQAPGQCLKWMGFINTATGEPTYADDFKGRFVFSLDTSVSTAYLQISSLKAEDTAVYFCTSGVHWGQGTLVTVSSGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 692 aa, synthetic construct
QVQLVQSGSELKKPGASVKVSCKASGYIFTDYAIHWVKQAPGQCLKWMGFINTATGEPTYADDFKGRFVFSLDTSVSTAYLQISSLKAEDTAVYFCTSGVHWGQGTLVTVSSGGGGSGGGGSGGGGSDIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGCGTKVEIKGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 693 aa, synthetic construct
QVQLVQSGSELKKPGASVKVSCKASGYIFTDYAIHWVKQAPGQGLKWMGFINTATGEPTYADDFKGRFVFSLDTSVSTAYLQISSLKAEDTAVYFCTSGVHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSS

SEQ ID NO: 30 - protein, 693 aa, synthetic construct
QVQLVQSGSELKKPGASVKVSCKASGYIFTDYAIHWVKQAPGQGLKWMGFINTATGEPTYADDFKGRFVFSLDTSVSTAYLQISSLKAEDTAVYFCTSGVHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 31 - protein, 696 aa, synthetic construct
QVQLVQSGSELKKPGASVKVSCKASGYIFTDYAIHWVKQAPGQGLKWMGFINTATGEPTYADDFKGRFVFSLDTSVSTAYLQISSLKAEDTAVYFCTSGVHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSS

SEQ ID NO: 32 - protein, 696 aa, synthetic construct
QVQLVQSGSELKKPGASVKVSCKASGYIFTDYAIHWVKQAPGQGLKWMGFINTATGEPTYADDFKGRFVFSLDTSVSTAYLQISSLKAEDTAVYFCTSGVHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 33 - protein, 214 aa, synthetic construct
DIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 213 aa, synthetic construct
DIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 462 aa, synthetic construct
DIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGCGTKVEIKGGGGSGGGGSGGGGSQVQLVQSGSELKKPGASVKVSCKASGYIFTDYAIHWVKQAPGQCLKWMGFINTATGEPTYADDFKGRFVFSLDTSVSTAYLQISSLKAEDTAVYFCTSGVHWGQGTLVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 462 aa, synthetic construct
QVQLVQSGSELKKPGASVKVSCKASGYIFTDYAIHWVKQAPGQCLKWMGFINTATGEPTYADDFKGRFVFSLDTSVSTAYLQISSLKAEDTAVYFCTSGVHWGQGTLVTVSSGGGGSGGGGSGGGGSDIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGCGTKVEIKGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 462 aa, synthetic construct
DIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSS

SEQ ID NO: 38 - protein, 462 aa, synthetic construct
DIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 39 - protein, 707 aa, synthetic construct
EVQLQESGPGLVKPSQTLSLTCTVSGYSITSGYTWHWIRQFPGNCLEWIGHIQYTGNTKYNPSLESRITISRDTSKNQFSLKLSSVTAADTATYYCARGSTNSNDYWGQGTLVTVSSGGGGSGGGGSGGGGSDIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGCGTKLELKGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 704 aa, synthetic construct
EVQLQESGPGLVKPSQTLSLTCTVSGYSITSGYTWHWIRQFPGNCLEWIGHIQYTGNTKYNPSLESRITISRDTSKNQFSLKLSSVTAADTATYYCARGSTNSNDYWGQGTLVTVSSGGGGSGGGGSGGGGSDIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGCGTKLELKGGGGSGGGGSGGGGSQVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 701 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 42 - protein, 698 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 43 - protein, 444 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 331 aa, synthetic construct
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 214 aa, synthetic construct; identical to SEQ ID NO: 33

SEQ ID NO: 46 - protein, 220 aa, synthetic construct
DIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 469 aa, synthetic construct
DIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQESGPGLVKPSETLSLTCTVSGSSLTSYGVHWVRQPPGKCLEGLGVIWPGGSTNYNSALMSRVTISKDNSKSQVSLKMSSLTAADTAVYYCARVTGTWYFDVWGQGTTVTVSSGGGGSGGGGSGGGGSDIQMTQSPSSLSASLGDRVTISCSASQGISNYLNWYQQKPDGTVKLLIYYTSTLHSGVPSRFSGSGSGTDYTLTISSLQPEDIATYYCQQYSKLPWTFGCGTKLEIK

SEQ ID NO: 48 - protein, 442 aa, synthetic construct
QVQLVQSGSELKKPGASVKVSCKASGYIFTDYAIHWVKQAPGQGLKWMGFINTATGEPTYADDFKGRFVFSLDTSVSTAYLQISSLKAEDTAVYFCTSGVHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 213 aa, synthetic construct; identical to SEQ ID NO: 34

SEQ ID NO: 50 - protein, 447 aa, synthetic construct
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 220 aa, synthetic construct; identical to SEQ ID NO: 46

SEQ ID NO: 52 - protein, 447 aa, synthetic construct
QVQLQESGPGLVKPSETLSLTCTVSGSSLTSYGVHWVRQPPGKGLEGLGVIWPGGSTNYNSALMSRVTISKDNSKSQVSLKMSSLTAADTAVYYCARVTGTWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 214 aa, synthetic construct; identical to SEQ ID NO: 33